(19) United States
(12) Patent Application Publication
(10) Pub. No.: US 2026/0108635 A1
(43) Pub. Date: Apr. 23, 2026

(54) ETHER-CONTAINING RADIOPAQUE POLYAMINO CROSSLINKERS AND MEDICAL HYDROGELS FORMED THEREFROM

(71) Applicant: Boston Scientific Scimed, Inc., Maple Grove, MN (US)

(72) Inventors: Rick William Dorn, Plymouth, MN (US); Cristian Parisi, Amherst, MA (US); Joseph Thomas Delaney, JR., Minneapolis, MN (US); Yen-Hao Hsu, Shrewsbury, MA (US)

(73) Assignee: Boston Scientific Scimed, Inc., Maple Grove, MN (US)

(21) Appl. No.: 19/364,325

(22) Filed: Oct. 21, 2025

Related U.S. Application Data
(60) Provisional application No. 63/710,311, filed on Oct. 22, 2024.

(51) Int. Cl. A61K 49/04; C08G 69/10

(52) U.S. Cl. CPC A61K 49/0442; A61K 49/0457; C08G 69/10

(57) ABSTRACT

In some aspects, the present disclosure provides iodinated polyamino compounds that comprise a polyamino moiety that is coupled to an iodinated aromatic moiety through a linkage that comprises an ether group, and optionally an amide group. Other aspects of the present disclosure pertain to systems for forming hydrogels that comprise (a) an iodinated polyamino compound that comprises a polyamino moiety that is coupled to an iodinated aromatic moiety through a linkage that comprises an ether group, and optionally an amide group, and (b) a reactive polymer that forms crosslinks with the iodinated polyamino compound. Still other aspects of the present disclosure pertain to crosslinked products formed from such systems and methods of treatment using such systems.

### CROSS-REFERENCE TO RELATED APPLICATIONS

This application claims the benefit of U.S. Provisional Patent Application Ser. No. 63/710,311 filed on Oct. 22, 2024, the disclosure of which is incorporated herein by reference.

### FIELD

The present disclosure relates to iodinated polyamino compounds, to hydrogels formed from iodinated polyamino compounds, and to methods of making and using iodinated polyamino compounds. The iodinated polyamino compounds of the present disclosure are useful, for example, in forming hydrogels for various biomedical applications.

### BACKGROUND

Bioresorbable hydrogels with rapid crosslinking reaction rates in vivo, known by the trade name of SpaceOAR®, have become a prominent biomaterial and obtained clinical success in creating the space between prostate and rectum, tremendously improving patient safety during the cancer therapies. SpaceOAR® is based on a multi-arm polyethylene glycol (PEG) polymer with a polyol core functionalized with succinimidyl glutarate (SG) as reactive end groups which further react with trilysine to form crosslinks. A further improvement based on this application is that some of 8-Arm PEG branches are functionalized with 2,3,5-triiiodobenzamide (TIB) groups, replacing part of the SG groups, in order to provide intrinsic radiopacity to the hydrogels themselves for CT-visibility. This hydrogel is known by the trade name of SpaceOAR Vue®. The hydrogels break down in-vivo over the course of ca. 6-9 months. The breakdown occurs primarily through the hydrolysis of the ester linkages on the glutarate groups.

In the event that water stability of the covalent bond connecting the trilysine to the iodinated species may be desired, an amide linkage would be preferred over an ester linkage. However, many iodinated species, and especially commercialized and approved iodinated contrast agents, contain primary alcohols, which can be used to form ester linkages, rather than primary amines, which would be useful in forming amide linkages. Nonetheless, inclusion of these approved iodinated contrast agents is desired as they are readily available, manufactured on large scales (which reduces cost) and well-studied from a toxicology/biological standpoint.

There is a continuing need in the biomedical arts for additional hydrogels, for precursors of such hydrogels, for methods of making such hydrogels and precursors, for methods of using such hydrogels and precursors, and for systems for forming such hydrogels, among other needs.

### SUMMARY

In various aspects, the present disclosure provides iodinated polyamino compound that comprise a polyamino moiety that is coupled to an iodinated aromatic moiety through a linkage that comprises an ether group. A linkage that comprises an ether group, like the above-noted amide linkage, provides more water stability compared to a linkage that comprises an ester group. In some embodiments, the linkage further comprises an amide group.

In some embodiments, which can be used in conjunction with the above aspects and embodiments, the iodinated aromatic moiety comprises a monocyclic or multicyclic aromatic structure that is substituted with one or more iodine atoms. In some of these embodiments, the monocyclic or multicyclic aromatic structure is further substituted with one or more C1-C4-hydroxyalkyl groups, which may comprise one or more vicinal-diol-containing groups in same cases. In some of these embodiments, at least one of the one or more C1-C4-hydroxy-containing groups is linked to the monocyclic or multicyclic aromatic structure through a linkage that comprise an amide group.

In some embodiments, which can be used in conjunction with the above aspects and embodiments, the polyamino moiety comprises a plurality of primary amine groups.

In some embodiments, which can be used in conjunction with the above aspects and embodiments, the iodinated polyamino compound comprises at least one residue of an iodinated aromatic compound that comprises one or more C1-C4-hydroxyalkyl groups and at least one residue of a poly(amino acid) that comprises a plurality of primary-amine-containing side groups. In some of these embodiments, the iodinated aromatic compound that comprises one or more C1-C4-hydroxyalkyl groups is selected from iopromide, metrizamide, iopamidol, iomeprol, ioxilan, iopentol, ioversol, iohexol, iobitridol, iodixanol, 5-acetamido-N,N′-bis(2,3-dihydroxypropyl)-2,4,6-triiodoisophthalamide, 5-(acetylamino)-N1,N3-bis(2,3-dihydroxypropyl)-2,4,6-triiodo-N1-methyl-1,3-benzenedicarboxamide, N1,N3-bis[2-hydroxy-1-(hydroxymethyl)ethyl]-5-[(2-hydroxy-1-oxopropyl)amino]-2,4,6-triiodo-1,3-benzenedicarboxamide, and 5,5′-[(1,3-dioxo-1,3-propanediyl)bis(methylimino)]bis[N,N′-bis[2,3-dihydroxy-1-(hydroxymethyl) propyl]-2,4,6-triiodo-1,3-benzenedicarboxamide. In some of these embodiments, the poly(amino acid) comprises a plurality of lysine residues.

In some embodiments, which can be used in conjunction with the above aspects and embodiments, the iodinated polyamino compound is formed by a method that comprises a nucleophilic substitution reaction between a haloalkyl-containing polyamino compound that comprises a plurality of primary amine groups and a C1-C4-haloalkyl group selected from a C1-C4-bromoalkyl group and a C1-C4-iodoalkyl group and a hydroxyalkyl-containing iodinated aromatic compound that comprises one or more C1-C4-hydroxyalkyl groups. In some of these embodiments, the haloalkyl-containing polyamino compound is formed by converting a C1-C4-carboxylic acid group of a carboxylic-acid-containing polyamino compound that comprises the plurality of primary amine groups and the C1-C4-carboxylic-acid group to either the C1-C4-bromoalkyl group or the C1-C4-iodoalkyl group. In some of these embodiments, the haloalkyl-containing polyamino compound is formed by converting a carboxylic acid group at a C-terminus of a poly(amino acid) compound that comprises the plurality of primary amine side groups to a hydroxymethyl group.

In some embodiments, which can be used in conjunction with the above aspects and embodiments, the iodinated polyamino compound is formed by a method that comprises a nucleophilic substitution reaction between a hydroxyalkyl-containing polyamino compound that comprises a plurality of primary amine groups and a C1-C4-hydroxyalkyl group and a haloalkyl-containing iodinated aromatic compound that comprises a C1-C4-haloalkyl group selected from a C1-C4-bromoalkyl group and a C1-C4-iodoalkyl group. In some of these embodiments, the hydroxyalkyl-containing polyamino compound is formed by reducing a C1-C4-carboxylic-acid group of a polyamino compound that comprises the plurality of primary amine groups and the C1-C4-carboxylic-acid group to the C1-C4-hydroxyalkyl group. In some of these embodiments, the hydroxyalkyl-containing polyamino compound is formed by reducing a carboxylic acid group at a C-terminus of a poly(amino acid) compound that comprises the plurality of primary amine side groups to a hydroxymethyl group. In some of these embodiments, the haloalkyl-containing iodinated aromatic compound comprising the C1-C4-haloalkyl group is formed by converting at least one hydroxyl group of an iodinated aromatic compound comprising a C1-C4-hydroxyalkyl group to either a bromine group or an iodine group.

In some embodiments, which can be used in conjunction with the above aspects and embodiments, the iodinated polyamino compound is formed by a method that comprises (a) reacting an iodinated aromatic compound comprising a C1-C4-hydroxyalkyl group in a nucleophilic substitution reaction with a halogen-containing amino compound that comprises at least one C1-C6-haloalkyl group and a protected amine group, where the a C1-C6-haloalkyl group is selected from a C1-C6-bromoalkyl group and a C1-C6-iodoalkyl group, (b) deprotecting the protected amine group and (c) reacting the deprotected amine group of the product of step (b) in an amide coupling reaction with a carboxylic acid group at a C-terminus of a poly(amino acid) compound that comprises a plurality of primary amine side groups.

In some embodiments, which can be used in conjunction with the above aspects and embodiments, the primary amine groups comprise —(CH₂)ₓ—NH₂ groups where x is 1, 2 3, 4, 5 or 6.

In some embodiments, which can be used in conjunction with the above aspects and embodiments, at least a portion of the primary amine groups are provided by lysine residues.

Other aspects of the present disclosure pertain to systems for forming hydrogels that comprise (a) an iodinated polyamino compound in accordance with the above aspects and embodiments and (b) a reactive polymer that forms crosslinks with the iodinated polyamino compound.

In some embodiments, the reactive polymer that forms crosslinks with the iodinated polyamino compound is a reactive multi-arm polymer that comprises a plurality of hydrophilic polymer arms comprising hydrophilic polymer segments and reactive end groups that covalently crosslink with primary amine groups of the iodinated polyamino compound. In some of these embodiments, the hydrophilic polymer segments are selected from polyalkylene oxide segments, polyester segments, polyoxazoline segments, polydioxanone segments, and polypeptide segments and/or the reactive groups are electrophilic groups selected from imidazole esters, imidazole carboxylates, benzotriazole esters, or imide esters. In some embodiments, the electrophilic groups are cyclic imide ester groups.

Other aspects of the present disclosure pertain to medical hydrogels that are formed by crosslinking an iodinated polyamino compound in accordance with the above aspects and embodiments with a reactive polymer that forms covalent crosslinks with the iodinated polyamino compound.

Still other aspects of the present disclosure pertain to methods of treatment comprising administering to a subject a mixture that comprises an iodinated polyamino compound in accordance with the above aspects and embodiments and a reactive polymer that forms covalent crosslinks with the iodinated polyamino compound under conditions such that the iodinated polyamino compound and the polymer form covalent crosslinks after administration.

Potential benefits associated with the present disclosure include one or more of the following: radiocontrast and crosslink density are optimized, and in vivo persistence is maintained as expected.

The above and other aspects, embodiments, features and benefits of the present disclosure will be readily apparent from the following detailed description.

### BRIEF DESCRIPTION OF THE DRAWINGS

FIG. 1A schematically illustrates a method in which a boc-protected trilysine is reacted with sodium borohydride followed by reaction with hypophosphorous tetraiodide to form a boc-protected iodoalkyl-substituted trilysine derivative, in accordance with an embodiment of the present disclosure.

FIG. 1B schematically illustrates a method in which the boc-protected iodoalkyl-substituted trilysine derivative of FIG. 1A is reacted with iodixanol in the presence of a potassium carbonate, followed by removal of the boc protection by exposure to acid, to yield an iodinated polyamino compound, in accordance with an embodiment of the present disclosure.

FIG. 2 schematically illustrates a method in which boc-protected trilysine is first reacted with sodium borohydride to form a boc-protected hydroxymethyl-substituted trilysine derivative, which is then is reacted with 1-(bromomethyl)-2,3,5-triiodobenzene, followed by removal of the boc protection, to yield an iodinated polyamino compound, in accordance with an embodiment of the present disclosure.

FIG. 3 schematically illustrates a method in which iopamidol is first partially acetal-protected, followed by conversion of the unprotected hydroxyl group to a bromine group using triphenylphosphine and carbon tetrabromide to form an acetal-protected brominated iopamidol derivative, which is then reacted with a boc-protected hydroxymethyl-substituted trilysine derivative in the presence of potassium carbonate, followed by removal of the acetal and Boc protection, to yield an iodinated polyamino compound, in accordance with an embodiment of the present disclosure.

FIG. 4 schematically illustrates a method in which iodixanol is reacted boc-protected 3-bromo-propylamine in the presence of potassium carbonate, followed by removal of the boc protection, to yield an iodinated amino compound, which is then reacted with boc-protected trilysine, in the presence of EDC as a coupling agent, followed by removal of the boc protection, to yield an iodinated polyamino compound, in accordance with an embodiment of the present disclosure.

FIG. 5A schematically illustrates a method in which acetal-protected iopamidol is reacted with boc-protected 1,5-dibromo-3-aminopentane, followed by removal of the boc protection, to yield an iodinated amino compound, which is reacted with boc-protected trilysine, in the presence of EDC as a coupling agent, followed by removal of the boc protection to yield an iodinated polyamino compound, in accordance with an embodiment of the present disclosure.

FIG. 5B schematically illustrates a method in which iopamidol is reacted with 2,2-dimethoxypropane to obtain acetal-protected iopamidol, in accordance with an embodiment of the present disclosure.

FIG. 5C schematically illustrates a method in which boc-protected 3-aminopentane, 1,5-dicarboxylic acid treated with sodium borohydride to form boc-protected 1,5-dihyoxy-3-aminopentane, which is then reacted with triphenylphosphine and carbon tetrabromide to form boc-protected 1,5-dibromo-3-aminopentane, in accordance with an embodiment of the present disclosure.

FIG. 6 schematically illustrates a covalent crosslinking reaction between a succinimide ester group and amino (primary amine) group whereby an amide linking group is formed, in accordance with an embodiment of the present disclosure.

FIG. 7 illustrates a delivery device, in accordance with an embodiment of the present disclosure.

FIG. 8 illustrates a delivery device, in accordance with another embodiment of the present disclosure.

### DETAILED DESCRIPTION

In various aspects, the present disclosure pertains to iodinated polyamino compounds (i.e., compounds that comprise one or more iodine groups and a plurality of amino (—NH₂) groups) that comprise a polyamino moiety that is coupled to an iodinated aromatic moiety through an ether linkage. In some embodiments, the iodinated polyamino compounds comprise a polyamino moiety that is coupled to an iodinated aromatic moiety through both an ether linkage and an amide linkage. Such iodinated polyamino compounds are useful, for example, as crosslinking agents for hydrogel formation. Ether and/or amide linkages are advantageous in cases where enhanced water stability of the coupling between the polyamino moiety and the iodinated aromatic moiety is desired.

In some embodiments, iodinated polyamino compounds in accordance with the present disclosure are prepared by a synthesis procedure in which an alcohol is reacted with a strong base such as sodium carbonate (Na₂CO₃), potassium carbonate (K₂CO₃), butyllithium (BuLi), potassium tert-butoxide (tBuOK), or sodium hydride (NaH) to form an alkoxide ion, which acts as a nucleophile that displaces a halide from an alkyl halide to yield an ether linkage.

In some embodiments, a haloalkyl-containing polyamino compound having one or more haloalkyl substituents selected from bromoalkyl substituents and iodoalkyl substituents, for instance C1-C6-haloalkyl substituents (e.g., independently selected from halomethyl, haloethyl, halopropyl, halobutyl, etc. substituents) is reacted with a hydroxyalkyl-containing iodinated aromatic compound having one or more hydroxyalkyl substituents (e.g., one or more primary or secondary alcohol substituents).

In some of these embodiments, a carboxylic acid group of an amine-protected carboxylic-acid-substituted polyamino compound is first converted to an haloalkyl group, preferably a bromoalkyl group or an iodoalkyl group, which can be reacted with the one or more hydroxyalkyl substituent(s) of a hydroxyalkyl-substituted iodinated aromatic compound.

Examples of amine-protective groups for use in the present disclosure include tert-butoxycarbonyl (boc) groups, carboxybenzyl (CBz) or (Z) groups, trifluoroacetyl (TFA) groups, and 9-fluorenylmethoxycarbonyl (Fmoc) groups, among others.

Conversion of the carboxylic acid group of the amine-protected carboxylic-acid-substituted polyamino compound to a haloalkyl group may be accomplished via multiple methods. In some embodiments, the amine-protected carboxylic-acid-substituted polyamino compound is treated with a reducing agent such as sodium borohydride followed by reaction with hypophosphorous tetraiodide (P₂I₄) or triphenylphosphine iodide as an iodine source, among other possibilities, to form an iodoalkyl group. Other reducing agents, such as lithium aluminum hydride, may be employed as well, depending on the stability of the protecting groups that are employed. Bromine-containing reagents, such as HBr, may be used to form a bromoalkyl group.

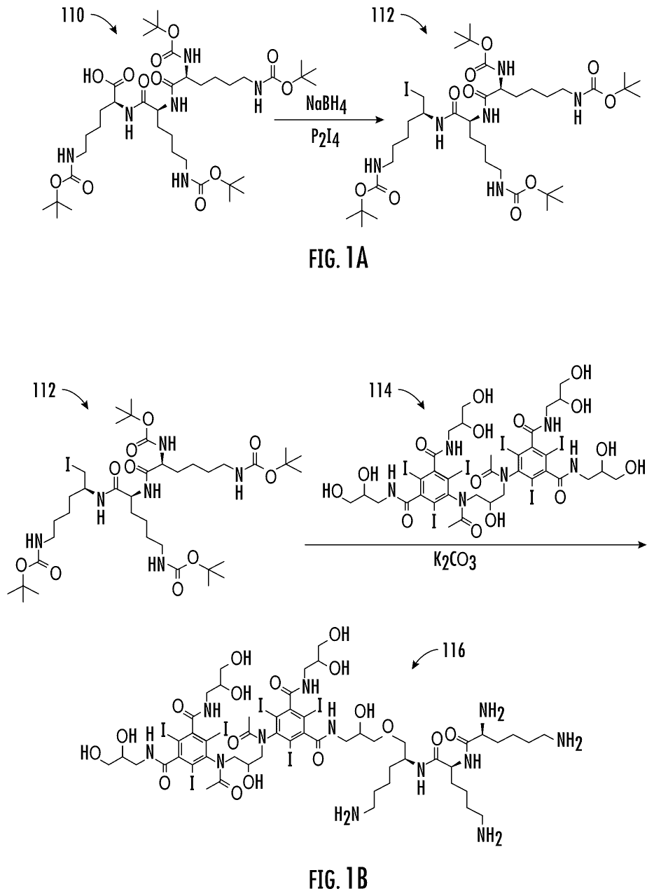

A particular embodiment in which boc-protected trilysine is employed as an amine-protected carboxylic-acid-substituted polyamino compound and iodixanol is employed as an iodinated aromatic compound having one or more hydroxyalkyl substituents is shown in FIGS. 1A-1B. In a first step, boc-protected trilysine (110) is reacted with sodium borohydride followed by reaction with hypophosphorous tetraiodide (P₂I₄) to form boc-protected haloalkyl-substituted trilysine derivative, specifically a boc-protected iodoalkyl-substituted trilysine derivative (112) in which an iodomethyl group is positioned at the position previously occupied by the carboxylic acid group of the boc-protected trilysine.

After forming an amine-protected haloalkyl-substituted polyamino compound from the amine-protected carboxylic-acid-substituted polyamino compound, the amine-protected haloalkyl-substituted polyamino compound is then reacted with a hydroxyalkyl-substituted iodinated aromatic compound in the presence of a strong base to yield an iodinated polyamino compound that contains an ether linkage between a residue of the carboxylic-acid-substituted polyamino compound and a residue of the hydroxyalkyl-substituted iodinated aromatic compound. In a particular embodiment shown in FIG. 1B, the boc-protected iodoalkyl-substituted trilysine derivative (112) of FIG. 1A is reacted with iodixanol (114) in the presence of a potassium carbonate, followed by removal of the boc protection, for example, by exposure to an acid such as HCl or trifluoroacetic acid, to yield an iodinated polyamino compound (116) in which an ether linkage is established between a trilysine residue and an iodixanol residue.

Where the hydroxyalkyl-substituted iodinated aromatic compound contains multiple hydroxyalkyl groups that are available for reaction, and an iodinated polyamino compound is desired in which only a single residue of the carboxylic-acid-substituted polyamino compound is linked to the residue of the hydroxyalkyl-substituted iodinated aromatic compound, a molar excess of the hydroxyalkyl-substituted iodinated aromatic compound relative to the amine-protected haloalkyl-substituted polyamino compound may be maintained. For example, with reference to FIG. 1B, an iodinated polyamino compound (116) in which only a single trilysine residue is attached the iodixanol residue may be produced by ensuring that a molar excess of the iodixanol (114) is maintained relative to the boc-protected iodoalkyl-substituted trilysine derivative (112). This may be achieved, for example, by dropwise addition of the boc-protected iodoalkyl-substituted trilysine derivative (112) to the iodixanol (114). This may also be achieved, for example, by combining a first solution having a first molar concentration of the boc-protected iodoalkyl-substituted trilysine derivative (112) with a second solution having a second molar concentration of the iodixanol (114), where the second molar concentration is higher than the first molar concentration. Yet another way this may be achieved is by using purification methods, such as recrystallization or preparative-scale chromatography, to select the iodinated polyamino compounds products that contain only one carboxylic-acid-substituted polyamino compound residue per hydroxyalkyl-substituted iodinated aromatic compound residue (e.g., only one trilysine residue per iodixanol residue as shown in FIG. 1B).

In other embodiments, a hydroxyalkyl-substituted polyamino compound having one or more hydroxyalkyl substituents (e.g., one or more primary or secondary alcohol substituents) is reacted with a haloalkyl-substituted iodinated aromatic compound having one or more haloalkyl substituents.

In some of these embodiments, a carboxylic acid group of an amine-protected carboxylic-acid-substituted polyamino compound is first converted to a hydroxyalkyl group, preferably a primary hydroxyalkyl group, which can then be reacted with the one or more haloalkyl substituents of the haloalkyl-substituted iodinated aromatic compound.

Conversion of the carboxylic acid group of the amine-protected carboxylic-acid-substituted polyamino compound to a hydroxyalkyl group may be accomplished via multiple methods. For example, the amine-protected carboxylic-acid-substituted polyamino compound may be treated with a reducing agent such as lithium aluminum hydride or sodium borohydride to reduce the carboxylic acid group to a primary hydroxyalkyl group depending on the protecting groups.

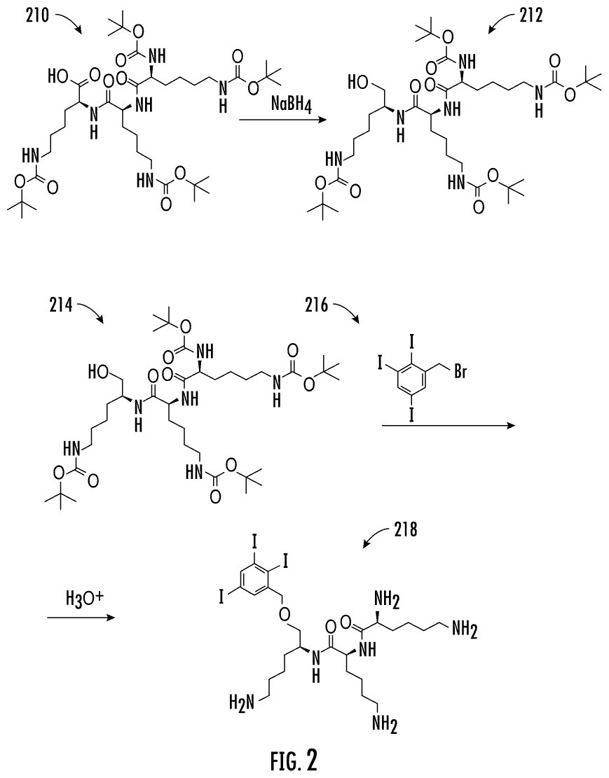

A particular embodiment in which boc-protected trilysine (210) is employed as an amine-protected carboxylic-acid-substituted polyamino compound and 1-(bromomethyl)-2,3,5-triiodobenzene (216) is employed as a haloalkyl-substituted iodinated aromatic compound is shown in FIG. 2. In a first step, boc-protected trilysine (210) is reacted with sodium borohydride (NaBH₄) to form a boc-protected hydroxyalkyl-substituted trilysine derivative, more specifically, a boc-protected hydroxymethyl-substituted trilysine derivative (212) in which a hydroxymethyl group is positioned at the position previously occupied by the carboxylic acid group of the boc-protected trilysine.

After forming an amine-protected hydroxyalkyl-substituted polyamino compound from the amine-protected carboxylic-acid-substituted polyamino compound, the amine-protected hydroxyalkyl-substituted polyamino compound is then reacted with a haloalkyl-substituted iodinated aromatic compound in the presence of a strong base to yield an iodinated polyamino compound that contains an ether linkage between a residue of the carboxylic-acid-substituted polyamino compound and a residue of the haloalkyl-substituted iodinated aromatic compound. In the particular embodiment shown in FIG. 2, the boc-protected hydroxymethyl-substituted trilysine derivative (212) is reacted with 1-(bromomethyl)-2,3,5-triiodobenzene (216) in the presence of a strong based such as potassium carbonate or sodium hydride, among others, followed by removal of the boc protection, for example, by exposure to an acid such as HCl or trifluoroacetic acid, to yield an iodinated polyamino compound (218) in which an ether linkage is established between a trilysine residue and a 1-(bromomethyl)-2,3,5-triiodobenzene residue.

In some embodiments, haloalkyl-substituted iodinated aromatic compounds may be formed from hydroxyalkyl-substituted iodinated aromatic compounds.

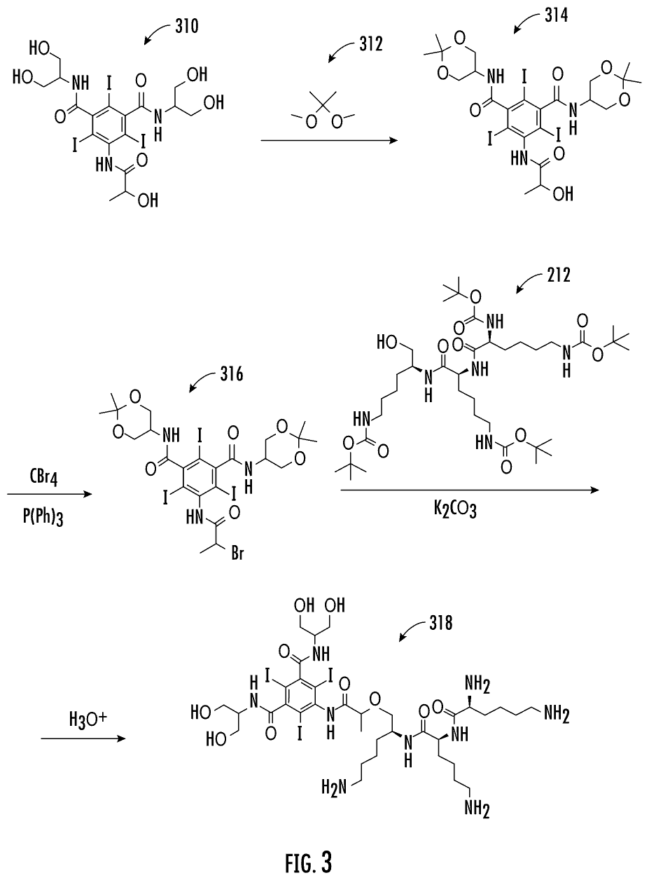

In some of these embodiments, a haloalkyl-substituted iodinated aromatic compound may be formed from a hydroxyalkyl-substituted iodinated aromatic compound by converting a hydroxyalkyl group of a hydroxyalkyl group-substituted iodinated aromatic compound to a bromoalkyl group. In a particular example shown in FIG. 3, the hydroxyalkyl group-substituted iodinated aromatic compound is iopamidol (310). In a first step shown in FIG. 3, the iopamidol (310) is partially protected such that only a single hydroxyl group remains unprotected. In particular, the vicinal diol groups of the iopamidol (310) are protected using 2,2-dimethoxypropane (312) in the presence of an organic solvent, such as dimethylformamide and p-toluenesulfonic acid (PTSA), to obtain an acetal-protected iopamidol (314) in which the non-vicinal-diol hydroxyl group remains unprotected. This unprotected hydroxyl group is then converted to a bromine group using triphenylphosphine (P(Ph)₃) and carbon tetrabromide (CBr₄) as a source of bromine atoms to convert the hydroxyalkyl group into a bromoalkyl group, thereby providing an acetal-protected brominated iopamidol derivative (316) in which the 2-hydroxypropanamido group of the iopamidol is converted to a 2-bromopropanamido group. The acetal-protected brominated iopamidol derivative (316) is then reacted with an amine-protected hydroxyalkyl-substituted polyamino compound, in particular, the boc-protected hydroxymethyl-substituted trilysine derivative (212) of FIG. 2, in the presence of potassium carbonate, followed by removal of the acetal and Boc protection, for example, by exposure to an acid such as HCl or trifluoroacetic acid, to yield an iodinated polyamino compound (318) in which an ether linkage is established between a trilysine residue and a iopamidol residue.

In other embodiments, the protection step may be skipped and all of the hydroxyl groups converted to halide groups, leading to the attachment of multiple amine-protected hydroxyalkyl-substituted polyamino compounds.

As noted above, in some embodiments of the present disclosure, iodinated polyamino compounds are formed that comprise a polyamino moiety that is coupled to an iodinated aromatic moiety through both an ether linkage and an amide linkage.

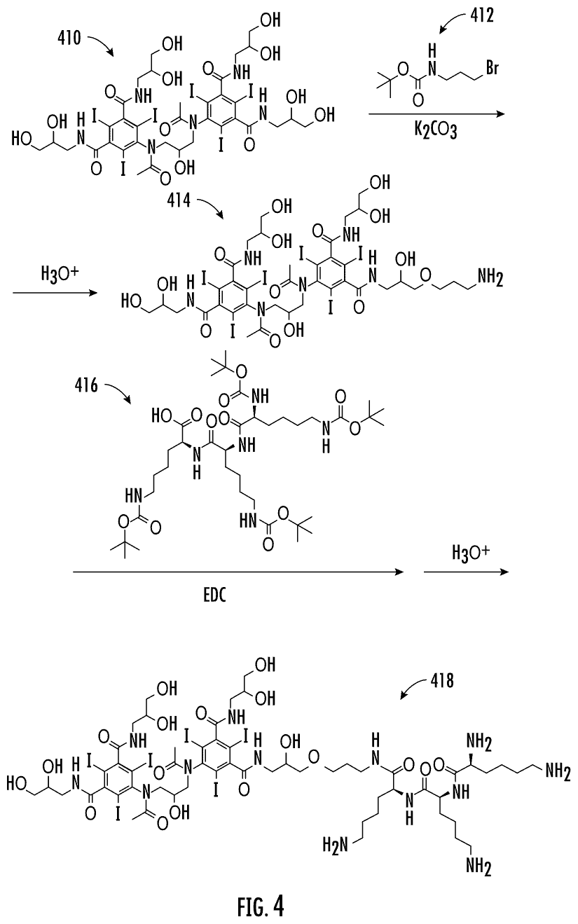

In some of these embodiments, an amino-substituted iodinated aromatic compound may first be formed from a hydroxyalkyl-substituted iodinated aromatic compound by reacting a hydroxyalkyl group of a hydroxyalkyl-substituted iodinated aromatic compound with an amine-protected halogen-containing amino compound, for example, a halogen-containing amino compound that comprises an alkyl portion substituted by an amino group and one or more halide groups. For example, with reference to FIG. 4, a hydroxyalkyl-substituted iodinated aromatic compound, specifically, iodixanol (410), may be reacted with an amine-protected halogen-containing amino compound, specifically, boc-protected 3-bromo-propylamine (412), which comprises propyl core substituted with an amino group and a bromo group, in the presence of a strong base, such as potassium carbonate, followed by removal of the boc protection, for example, by exposure to an acid such as HCl or trifluoroacetic acid, to yield an iodinated amino compound (414) in which a residue of the hydroxyalkyl-substituted iodinated aromatic compound is linked to an amino alkyl moiety through an ether linkage.

The amino group of the iodinated amino compound (414) may then be reacted with a carboxylic acid group of an amine-protected carboxylic-acid-substituted polyamino compound in the presence of a suitable amide coupling agent, to form an iodinated polyamino compound that comprises a polyamino moiety that is coupled to an iodinated aromatic moiety through both an ether linkage and an amide linkage. Examples of suitable amide coupling agents include carbodiimide coupling agents such as N,N′-dicyclohexylcarbodiimide (DCC), 1-ethyl-3-(3-dimethyl propyl) carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others. For example, in the particular embodiment shown in FIG. 4, the iodinated amino compound (414) is reacted with the carboxylic acid group of an amine-protected carboxylic-acid-substituted polyamino compound, specifically boc-protected trilysine (416), in the presence of EDC as a coupling agent, followed by removal of the boc protection, for example, by exposure to an acid such as HCl or trifluoroacetic acid, to yield an iodinated polyamino compound (418) that comprises a polyamino moiety that is coupled to an iodinated aromatic moiety through both an ether linkage and a newly formed amide linkage.

While only a single hydroxyl group of the hydroxyalkyl-substituted iodinated aromatic compound is reacted with the amine-protected halogen-containing amino compound in FIG. 4, in other embodiments, multiple hydroxyl groups may be reacted with the amine-protected halogen-containing amino compound, ultimately leading to attachment of multiple amine-protected carboxylic-acid-substituted polyamino compounds.

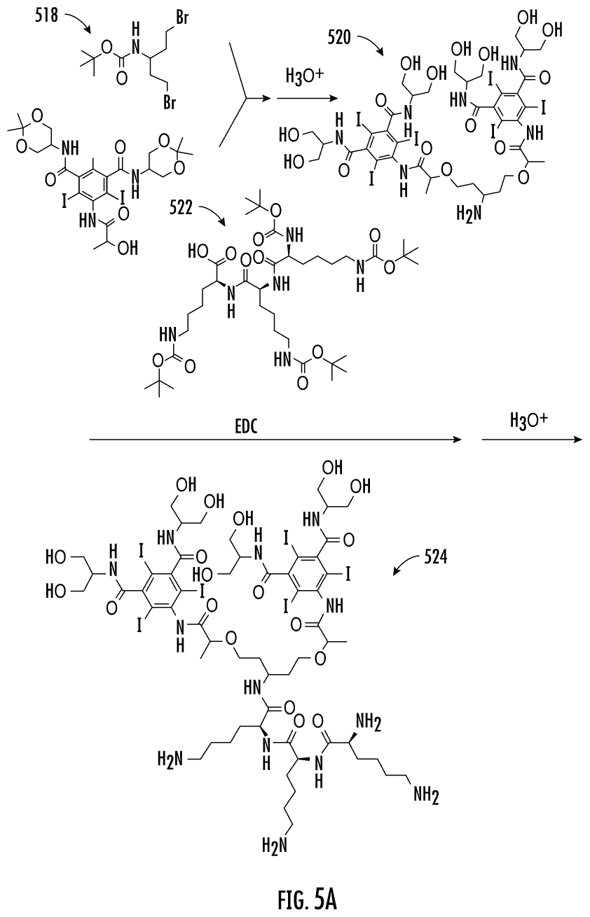

As another example, with reference to FIG. 5A, a hydroxyalkyl-substituted iodinated aromatic compound, specifically, acetal-protected iopamidol (512), may be reacted with an amine-protected, halogen-substituted aminoalkyl compound, specifically, tert-butyl N-[3-bromo-1-(2-bromoethyl) propyl]carbamate (518), in the presence of a strong base, such as potassium carbonate followed by removal of the boc protection, for example, by exposure to an acid such as HCl or trifluoroacetic acid, to yield an iodinated amino compound (520) in which two residues of the hydroxyalkyl-substituted iodinated aromatic compound are linked to an aminoalkyl moiety through an ether linkage.

The amino group of the iodinated amino compound may then be reacted with a carboxylic acid group of an amine-protected carboxylic-acid-substituted polyamino compound in the presence of a suitable amide coupling agent, to form an iodinated polyamino compound that comprises a polyamino moiety that is coupled to an iodinated aromatic moiety through both an ether linkage and an amide linkage. For example, in the particular embodiment shown in FIG. 5A, the iodinated amino compound (520) is reacted with the carboxylic acid group of an amine-protected carboxylic-acid-substituted polyamino compound, specifically boc-protected trilysine (522), in the presence of EDC as a coupling agent, followed by removal of the boc protection, for example, by exposure to an acid such as HCl or trifluoroacetic acid, to yield an iodinated polyamino compound (524) that comprises a polyamino moiety that is coupled to an iodinated aromatic moiety through both an ether linkage and a newly formed amide linkage.

One method of forming the acetal-protected iopamidol (512) of FIG. 5A is shown in FIG. 5B. With reference to this figure, the vicinal diol groups of a hydroxyalkyl-substituted iodinated aromatic compound, specifically, iopamidol (510), are protected using 2,2-dimethoxypropane in the presence of an organic solvent, such as dimethylformamide and p-toluenesulfonic acid (PTSA), to obtain an acetal-protected iopamidol (512).

One method of forming the tert-butyl N-[3-bromo-1-(2-bromoethyl) propyl]carbamate (518) of FIG. 5A is shown in FIG. 5C. With reference to FIG. 5C, an amine-protected polycarboxylic-acid-substituted aminoalkyl compound, specifically, 3-tert-butoxycarbonylaminopentanedioic acid (514), is treated with a reducing agent such as sodium borohydride to reduce the carboxylic acid groups of the boc-protected 3-aminopentane, 1,5-dicarboxylic acid (514) to hydroxymethyl groups, thereby forming an amine-protected polyhydroxy-substituted aminoalkyl compound, specifically, tert-butyl N-[3-hydroxy-1-(2-hydroxyethyl) propyl]carbamate (516). The hydroxyl groups are then converted to bromine groups using triphenylphosphine (P(Ph)₃) and carbon tetrabromide (CBr₄) as a source of bromine atoms, thereby providing a boc-protected halogen-substituted aminoalkyl compound, specifically, boc-protected 1,5-dibromo-3-aminopentane (518), which contains two primary bromine groups.

In various embodiments of the present disclosure, iodinated polyamino compounds of the present disclosure comprise a polyamino moiety having a plurality (two, three, four, five, six, seven, eight, nine, ten or more)amino groups. For example, the polyamino moiety may comprises a plurality (two, three, four, five, six, seven, eight, nine, ten or more) of −(CH₂)ₓ—NH₂ groups where x is 0, 1, 2 3, 4, 5 or 6. In some of these embodiments, the polyamino moiety may comprises a plurality of —(CH₂)ₓ—NH₂ groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, a polyalkylene moiety, or a polysaccharide moiety, among others.

Examples of carboxylic-acid-substituted polyamino compounds, also referred to herein as carboxylic-acid-containing polyamino compounds, which can be used to form iodinated polyamino compounds in accordance with the present disclosure include poly(amino acids) that comprise a plurality of primary-amine-containing side groups (e.g., 2, 3, 4, 5, 6, 7, 8, 9, 10 or more primary-amine-containing side groups), such as, for example, poly(amino acids) that comprise lysine and/or ornithine (e.g., polylysine compounds such as dilysine, trilysine, tetralysine, pentalysine, etc., polyornithine compounds such as diornithine, triornithine, tetraornithine, pentaornithine, etc., and poly(lysine-co-ornithine) compounds), as well as carboxylic-acid-terminated polyamines such as a carboxylic-acid-terminated poly(allyl amine), a carboxylic-acid-terminated poly(vinyl amine), or a carboxylic-acid-terminated chitosan.

Specific examples of iodinated moieties include those that comprise one or more monocyclic or multicyclic aromatic structures, substituted with (a) one or more iodine groups (e.g., one two, three, four, five, six, seven, eight, nine, ten or more iodine atoms) and (b) optionally, one or more hydroxyl-containing groups independently selected from one or more hydroxyl groups and/or one or more C1-C4-hydroxyalkyl groups (e.g., C1-C4-monohydroxyalkyl groups, C1-C4-dihydroxyalkyl groups, C1-C4-trihydroxyalkyl groups, C1-C4-tetrahydroxyalkyl groups, etc.), among others, which C1-C4-hydroxyalkyl groups may be linked to the monocyclic or multicyclic aromatic structures directly or through any suitable linking moiety, which may be selected, for example, from alkyl groups (e.g., alkyl groups containing one carbon, two carbons, three carbons, four carbons, etc.), amide groups, ether groups, urea groups, urethane groups, and combinations thereof, among others

In various embodiments, iodinated moieties of the present disclosure are residues of hydroxyalkyl-substituted iodinated aromatic compounds, also referred to herein as hydroxyalkyl-containing iodinated aromatic compounds. Specific examples of hydroxyalkyl-substituted iodinated aromatic compounds include those that comprise one or more monocyclic or multicyclic aromatic structures, substituted with (a) one or more iodine groups (e.g., one two, three, four, five, six, seven, eight, nine, ten or more iodine atoms) and (b) one or more hydroxyl-containing groups independently selected from one or more hydroxyl groups and/or one or more C1-C4-hydroxyalkyl groups (e.g., C1-C4-monohydroxyalkyl groups, C1-C4-dihydroxyalkyl groups, C1-C4-trihydroxyalkyl groups, C1-C4-tetrahydroxyalkyl groups, etc.), among others, which C1-C4-hydroxyalkyl groups may be linked to the monocyclic or multicyclic aromatic structures directly or through any suitable linking moiety, which may be selected, for example, from alkyl groups (e.g., alkyl groups containing one carbon, two carbons, three carbons, four carbons, etc.), amide groups, ether groups, urea groups, urethane groups, and combinations thereof, among others.

More specific examples of hydroxyalkyl-substituted iodinated aromatic compounds that can be used to form iodinated polyamino compounds in accordance with the present disclosure include those presented in the table below.

| | | # of OH |
|---|---|---|
| Compound | CAS# | Groups |
| Iopromide | 73334-07-3 | 4 |
| 5-Acetamido-N,N′-bis(2,3-dihydroxypropyl)- | 31127-80-7 | 4 |
| 2,4,6-triiodoisophthalamide | | |
| Metrizamide | 31112-62-6 | 4 |
| 1,3-Benzenedicarboxamide, 5-(acetylamino)- | 76350-28-2 | 4 |
| N1,N3-bis(2,3-dihydroxypropyl)-2,4,6-triiodo- | | |
| N1-methyl- (ACI) | | |
| Iopamidol | 60166-93-0 | 5 |
| Iomeprol | 78649-41-9 | 5 |
| 1,3-Benzenedicarboxamide, N1,N3-bis[2- | 60208-45-9 | 5 |
| hydroxy-1-(hydroxymethyl)ethyl]-5-[(2-hydroxy- | | |
| 1-oxopropyl)amino]-2,4,6-triiodo- (ACI) | | |
| Ioxilan | 107793-72-6 | 5 |
| Iopentol | 89797-00-2 | 5 |
| Ioversol | 87771-40-2 | 6 |
| Iohexol | 66108-95-0 | 6 |
| Iobitridol | 136949-58-1 | 6 |
| Iodixanol | 92339-11-2 | 9 |
| 1,3-Benzenedicarboxamide, 5,5′-[(1,3-dioxo-1,3- | 79770-24-4 | 12 |
| propanediyl)bis(methylimino)]bis[N,N′-bis[2,3- | | |
| dihydroxy-1-(hydroxymethyl)propyl]-2,4,6- | | |
| triiodo- (9CI, ACI) | | |

In other aspects of the present disclosure, crosslinked reaction products of (a) an iodinated polyamino compound such as those described above and (b) a reactive polymer having reactive groups that are reactive with the amino groups of the iodinated polyamino compound, for example, a reactive multi-arm polymer that comprises a plurality of polymer arms that have reactive end groups that are reactive with the amino groups of the iodinated polyamino compound, are provided.

In some embodiments, the crosslinked reaction products are hydrogels. As used herein, a “hydrogel,” which may also be referred to herein as a “crosslinked hydrogel,” is a crosslinked polymer that contains water or can absorb water but does not dissolve when placed in water.

In various embodiments, such crosslinked products are visible using X-ray imaging techniques. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on a bench-top micro CT system such as Xtreme CT from Scanco Medical (Wangen-Brüttisellen, Switzerland) or similar.

Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In various embodiments, the reactive end groups of the reactive multi-arm polymer and the amino groups of the iodinated polyamino compound react with one another via an amide coupling reaction to form a crosslinked product.

In some embodiments, reactive multi-arm polymers in accordance with the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a first reactive moiety is covalently attached to an opposite end of the hydrophilic polymer segment through a suitable linkage.

Reactive multi-arm polymers in accordance with the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

Reactive end groups include those that comprise electrophilic groups. Electrophilic groups may be selected, for example, from cyclic imide ester groups, such as succinimide ester groups,

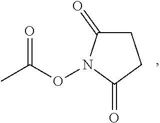

maleimide ester groups, glutarimide ester groups, diglycolimide ester groups, phthalimide ester groups, and bicyclo[2.2.1]hept-5-ene-2,3-dicarboxylic acid imide ester groups,

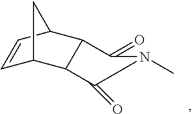

imidazole ester groups, imidazole carboxylate groups and benzotriazole ester groups, among other possibilities.

The electrophilic groups may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising three or more hydroxyl groups, also referred to herein as a “polyol”, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, nonamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl)alkanes, such as 1,1,1-tris(4-hydroxyphenyl) ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Reactive multi-arm polymers in accordance with the present disclosure can be formed from hydroxy-terminated multi-arm polymers having arms that comprise one or more hydroxyl end groups. In some embodiments of the present disclosure, a polyol such as one of those described below, among others, may be used as multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, USA.

In some embodiments, a hydroxy-terminated multi-arm hydrophilic polymer may be reacted with a cyclic anhydride to form carboxylic-acid-terminated polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

An electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In some aspects of the present disclosure, systems are provided that are configured to deliver (a) an iodinated polyamino compound in accordance with the present disclosure and (b) a reactive multi-arm polymer as described herein. The iodinated polyamino compound and the reactive multi-arm polymer are combined under conditions such that the amino groups of the iodinated polyamino compound and the reactive end groups of the reactive multi-arm polymer crosslink with one another. In certain embodiments, those conditions comprise an environment having a basic pH, for example, a pH ranging from about 8.5 to about 12. Such systems can be used to form crosslinked hydrogels, either in vivo or ex vivo.

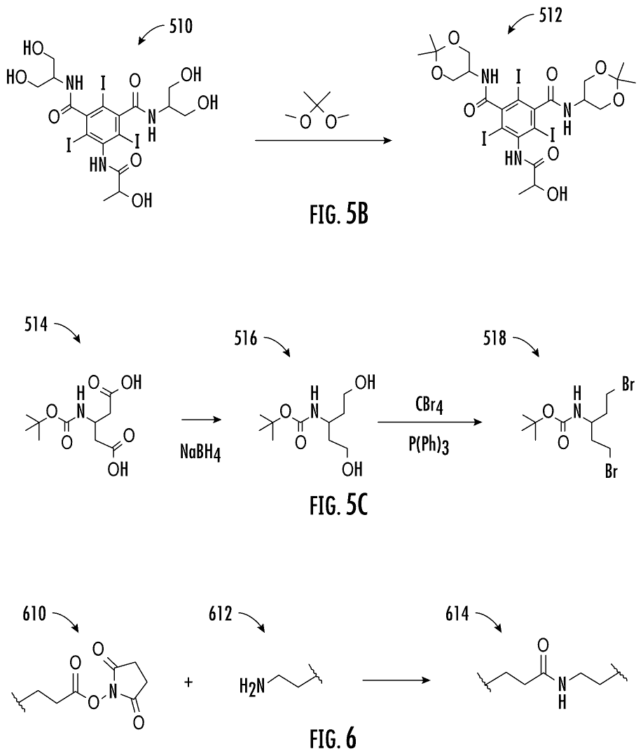

A particular example of a crosslinking reaction is illustrated in FIG. 6, which show a covalent crosslinking reaction between a cyclic amide ester group, specifically, a succinimide ester group of a reactive multi-arm polymer 610 as described herein and reactive amino group 612 of an iodinated polyamino compound as described herein, whereby an amide linking group 614 is formed. The crosslinking reaction shown is inhibited at acidic pH, but occurs spontaneously at basic pH.

In some aspects of the present disclosure, a system is provided that comprises (a) a first composition that comprises an iodinated polyamino as described herein and (b) a second composition that comprises a reactive multi-arm polymer as described herein.

The first composition may be a first fluid composition comprising the iodinated polyamino compound or a first dry composition that comprises the iodinated polyamino compound, to which a suitable fluid such as water for injection, saline, etc. can be added to form a first fluid composition. In addition to the iodinated polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including therapeutic agents, imaging agents, colorants, tonicity adjusting agents, suspension agents, wetting agents, and pH adjusting agents as described below.

In some embodiments, the iodinated polyamino compound is initially combined with the reactive multi-arm polymer under conditions where crosslinking between the electrophilic moieties of the reactive multi-arm polymer and the amino groups of the iodinated polyamino compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In particular embodiments, the system comprises (a) a first composition that comprises an iodinated polyamino compound as described hereinabove, (b) a second composition that comprises a reactive multi-arm polymer as described hereinabove, and (c) a third composition, specifically, an accelerant composition, that contains an accelerant that is configured to accelerate a crosslinking reaction between the iodinated polyamino compound and the reactive multi-arm polymer.

The first composition may be a first fluid composition comprising the iodinated polyamino compound that is buffered to an acidic pH or a first dry composition that comprises the iodinated polyamino compound and acidic buffering composition, to which a suitable fluid such as water for injection, saline, etc. can be added to form a first fluid composition comprising the iodinated polyamino compound that is buffered to an acidic pH. In some embodiments, for example, the acidic buffering composition may comprise monobasic sodium phosphate, among other possibilities. The first fluid composition comprising the iodinated polyamino compound may have a pH ranging, for example, from about 3 to about 6.5. In addition to the iodinated polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the iodinated polyamino compound that is buffered to an acidic pH. In addition to the reactive multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the iodinated polyamino compound that is buffered to an acidic pH and the second composition comprises a dry composition that comprises the reactive multi-arm polymer. The first composition may then be mixed with the second composition to provide a prepared fluid composition that is buffered to an acidic pH and comprises the iodinated polyamino compound and the reactive multi-arm polymer. In a particular example, a syringe may be provided that contains the first fluid composition comprising the iodinated polyamino compound that is buffered to an acidic pH, and a vial may be provided that comprises the dry composition (e.g., a powder) that comprises the reactive multi-arm polymer. The syringe may then be used to inject the first fluid composition into the vial containing the reactive multi-arm polymer to form a prepared fluid composition that contains the iodinated polyamino compound and the reactive multi-arm polymer, which can be withdrawn back into the syringe for administration.

The third composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 11.5. In addition to the above, the fluid accelerant composition may further comprise additional agents, including those described below.

A prepared fluid composition that is buffered to an acidic pH and comprises the iodinated polyamino compound and the reactive multi-arm polymer as described above (as well as additional agents in some cases), and a fluid accelerant composition that is buffered to basic pH as described above (which may include additional agents in some cases), may be combined form crosslinked hydrogels, either in vivo or ex vivo.

In some particular embodiments of the present disclosure, a kit is provided that include a first reservoir (e.g., a vial or syringe barrel) containing a first composition comprising an iodinated polyamino compound as described herein and an acidic buffer, a second reservoir (e.g., a vial or syringe barrel) containing a second composition comprising containing a reactive multi-arm polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the iodinated polyamino compound and the reactive multi-arm polymer, and additional apparatus for combining and delivering the prepared fluid composition and buffered accelerant to a patient.

In more particular embodiments, the kit may comprise a vial containing a reactive multi-arm polymer as described herein in dry (e.g., powdered) form, a first syringe containing a fluid composition comprising an iodinated polyamino compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The compositions described herein may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxylic acid groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the iodinated polyamino compound and the reactive multi-arm polymer as described above, and a fluid accelerant composition that is buffered to basic pH as described above, may be combined form crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises an iodinated polyamino compound as described above and a second reservoir that contains a second composition that comprises a reactive multi-arm polymer that comprises a plurality of electrophilic moieties that are reactive with the amino moieties of the iodinated polyamino compound as described above.

In some embodiments, the system may include a delivery device that comprises a first reservoir that contains a first composition that comprises the iodinated polyamino compound and the reactive multi-arm polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the iodinated polyamino compound and the reactive multi-arm polymer and crosslink with one another to form a crosslinked hydrogel.

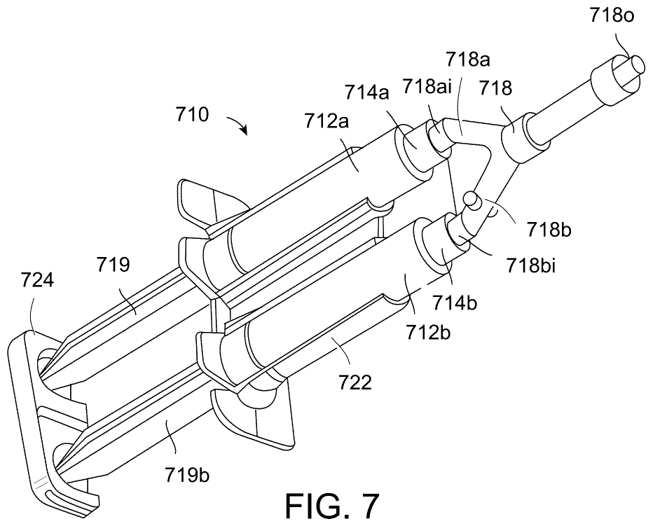

In particular embodiments, and with reference to FIG. 7, the system may include a delivery device 710 that comprises a double-barrel syringe, which includes a first barrel 712a having a first barrel outlet 714a, which first barrel contains a first fluid composition as described above, a first plunger 719a that is movable in the first barrel 712a, a second barrel 712b having a second barrel outlet 714b, which second barrel 712b contains a second fluid composition as described above, and a second plunger 719b that is movable in the second barrel 712b. In some embodiments, the device 710 may further comprise a mixing section 718 (e.g., a Y-connector) having a first mixing section inlet 718ai in fluid communication with the first barrel outlet 714a, a second mixing section inlet 718bi in fluid communication with the second barrel outlet 714b, and a mixing section outlet 7180. Also shown are a syringe holder 722 configured to hold the first and second syringe barrels 712a, 712b, in a fixed relationship and a plunger cap 724 configured to hold the first and second plungers 719a, 719b in a fixed relationship.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

The crosslinked hydrogel compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

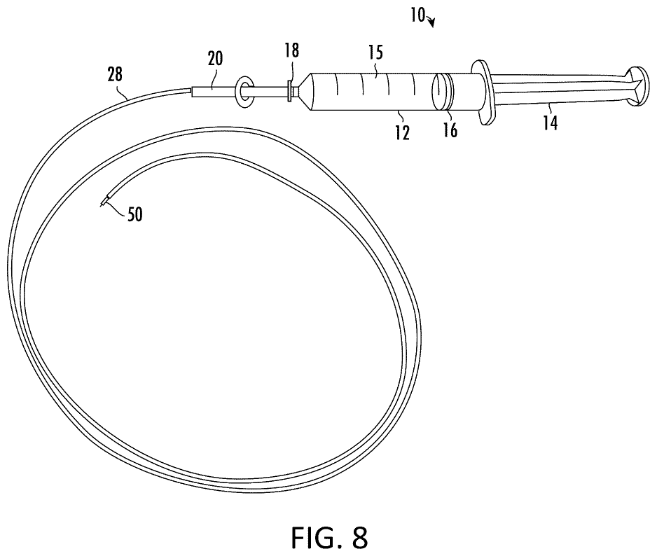

FIG. 8 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

During and/or after administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. An iodinated polyamino compound comprising a polyamino moiety that is coupled to an iodinated aromatic moiety through a linkage comprising an ether group.

2. The iodinated polyamino compound of claim 1, wherein the linkage further comprises an amide group.

3. The iodinated polyamino compound of claim 1, wherein the iodinated aromatic moiety comprises a monocyclic or multicyclic aromatic structure that is substituted with one or more iodine atoms.

4. The iodinated polyamino compound of claim 3, wherein the monocyclic or multicyclic aromatic structure is further substituted with one or more C1-C4-hydroxyalkyl groups.

5. The iodinated polyamino compound of claim 3, wherein the monocyclic or multicyclic aromatic structure is further substituted with one or more C1-C4-hydroxyalkyl groups that comprise one or more vicinal-diol-containing groups.

6. The iodinated polyamino compound of claim 4, wherein at least one of the one or more C1-C4-hydroxy-containing groups is linked to the monocyclic or multicyclic aromatic structure through a linkage that comprise an amide group.

7. The iodinated polyamino compound of claim 1, wherein the polyamino moiety comprises a plurality of primary amine groups.

8. The iodinated polyamino compound of claim 1, wherein the iodinated polyamino compound comprises at least one residue of an iodinated aromatic compound that comprises one or more C1-C4-hydroxyalkyl groups and at least one residue of a poly(amino acid) that comprises a plurality of primary-amine-containing side groups.

9. The iodinated polyamino compound of claim 8, wherein the iodinated aromatic compound that comprises one or more C1-C4-hydroxyalkyl groups is selected from iopromide, metrizamide, iopamidol, iomeprol, ioxilan, iopentol, ioversol, iohexol, iobitridol, iodixanol, 5-acetamido-N,N′-bis(2,3-dihydroxypropyl)-2,4,6-triiodoisophthalamide, 5-(acetylamino)-N1,N3-bis(2,3-dihydroxypropyl)-2,4,6-triiodo-N1-methyl-1,3-benzenedicarboxamide, N1,N3-bis[2-hydroxy-1-(hydroxymethyl)ethyl]-5-[(2-hydroxy-1-oxopropyl)amino]-2,4,6-triiodo-1,3-benzenedicarboxamide, and 5,5′-[(1,3-dioxo-1,3-propanediyl)bis(methylimino)]bis[N,N′-bis[2,3-dihydroxy-1-(hydroxymethyl) propyl]-2,4,6-triiodo-1,3-benzenedicarboxamide.

10. The iodinated polyamino compound of claim 8, wherein the poly(amino acid) comprises a plurality of lysine residues.

11. The iodinated polyamino compound of claim 1, wherein the iodinated polyamino compound is formed by a method that comprises a nucleophilic substitution reaction between a haloalkyl-containing polyamino compound that comprises a plurality of primary amine groups and a C1-C4-haloalkyl group selected from a C1-C4-bromoalkyl group and a C1-C4-iodoalkyl group and a hydroxyalkyl-containing iodinated aromatic compound that comprises one or more C1-C4-hydroxyalkyl groups.

12. The iodinated polyamino compound of claim 11, wherein the haloalkyl-containing polyamino compound is formed by converting a C1-C4-carboxylic acid group of a carboxylic-acid-containing polyamino compound that comprises the plurality of primary amine groups and the C1-C4-carboxylic-acid group to either the C1-C4-bromoalkyl group or the C1-C4-iodoalkyl group.

13. The iodinated polyamino compound of claim 11, wherein the haloalkyl-containing polyamino compound is formed by converting a carboxylic acid group at a C-terminus of a poly(amino acid) compound that comprises the plurality of primary amine side groups to a hydroxymethyl group.

14. The iodinated polyamino compound of claim 1, wherein the iodinated polyamino compound is formed by a method that comprises a nucleophilic substitution reaction between a hydroxyalkyl-containing polyamino compound that comprises a plurality of primary amine groups and a C1-C4-hydroxyalkyl group and a haloalkyl-containing iodinated aromatic compound that comprises a C1-C4-haloalkyl group selected from a C1-C4-bromoalkyl group and a C1-C4-iodoalkyl group.

15. The iodinated polyamino compound of claim 14, wherein the hydroxyalkyl-containing polyamino compound is formed by reducing a C1-C4-carboxylic-acid group of a polyamino compound that comprises the plurality of primary amine groups and the C1-C4-carboxylic-acid group to the C1-C4-hydroxyalkyl group.

16. The iodinated polyamino compound of claim 14, wherein the hydroxyalkyl-containing polyamino compound is formed by reducing a carboxylic acid group at a C-terminus of a poly(amino acid) compound that comprises the plurality of primary amine side groups to a hydroxymethyl group.

17. The iodinated polyamino compound of claim 14, where the haloalkyl-containing iodinated aromatic compound comprising the C1-C4-haloalkyl group is formed by converting at least one hydroxyl group of an iodinated aromatic compound comprising a C1-C4-hydroxyalkyl group to either a bromine group or an iodine group.

18. The iodinated polyamino compound of claim 1, wherein the iodinated polyamino compound is formed by a method that comprises (a) reacting an iodinated aromatic compound comprising a C1-C4-hydroxyalkyl group in a nucleophilic substitution reaction with a halogen-containing amino compound that comprises at least one C1-C6-haloalkyl group and a protected amine group, where the a C1-C6-haloalkyl group is selected from a C1-C6-bromoalkyl group and a C1-C6-iodoalkyl group, (b) deprotecting the protected amine group and (c) reacting the deprotected amine group of the product of step (b) in an amide coupling reaction with a carboxylic acid group at a C-terminus of a poly(amino acid) compound that comprises a plurality of primary amine side groups.

19. A system for forming a hydrogel that comprises an iodinated polyamino compound comprising a polyamino moiety that is coupled to an iodinated aromatic moiety through a linkage comprising an ether group and a reactive polymer that forms crosslinks with the iodinated polyamino compound.

20. A method of treatment comprising administering to a subject a mixture that comprises an iodinated polyamino compound comprising a polyamino moiety that is coupled to an iodinated aromatic moiety through a linkage comprising an ether group and a reactive polymer that forms covalent crosslinks with the iodinated polyamino compound under conditions such that the iodinated polyamino compound and the polymer form covalent crosslinks after administration.
